(19) United States
(12) Patent Application Publication
(10) Pub. No.: US 2024/0125774 A1
(43) Pub. Date: Apr. 18, 2024

(54) PASSIVE METHOD FOR IMPROVING SENSITIVITY OF BIOASSAY WITH SEGREGATIVE PHASE SEPARATION

(71) Applicants: The University of Hong Kong, Hong Kong (CN); Advanced Biomedical Instrumentation Centre Limited, Hong Kong (CN)

(72) Inventors: Jingxuan Tian, Hong Kong (CN); Ho Cheung Shum, Hong Kong (CN); Yang Cao, Hong Kong (CN); Hao Yuan, Hong Kong (CN)

(21) Appl. No.: 18/485,856

(22) Filed: Oct. 12, 2023

Related U.S. Application Data
(60) Provisional application No. 63/379,954, filed on Oct. 18, 2022.

(51) Int. Cl. G01N 33/542

(52) U.S. Cl. CPC G01N 33/542

(57) ABSTRACT

A bioassay, which is a segregative phase separation system (SPSS) where a bioassay signal intensifier is presented. The bioassay signal intensifier is an aqueous two-phase system (ATPS) component that causes the signal to intensify in one aqueous phase of a plurality of phases formed from the bioassay composition. The source of the signal resulting from the inclusion of a test sample is selectively partitioned into one phase that can be of significantly lesser volume than in the bioassay absent the bioassay signal intensifier, such the signal's source is concentrated in that phase, and the signal is intensified.

### CROSS-REFERENCE TO RELATED APPLICATION

This application claims the benefit of U.S. Provisional Application Ser. No. 63/379,954, filed Oct. 18, 2022, which is hereby incorporated by reference in its entirety including any tables, figures, or drawings.

### BACKGROUND OF THE INVENTION

Traditionally potency of biological products is by a biological assay (bioassay) that measures activity relative to its ability to affect a given result. Bioassays measure potency by evaluating a product's active ingredient in a living biological system. Bioassays can include in vivo animal studies, in vitro organ, tissue, or cell culture systems, or any combination of these. The sensitivity of a bioassay is a constant concern for assurance that the biological product is detected with a viable sample size in its inherent concentration. To assure that the assay is not adversely affected, passive methods are attractive for enhancing sensitivity.

Existing methods for improving bioassay sensitivity require extra input, usually an excessive amount that can involve more steps than the ones required for performing the assay. These active methods include introducing particles, such as nanorods or micro-beads, for binding the signal molecules and imposition of an electric field or magnetic field to manipulate and aggregate these particles. The additives and the extra instruments required can be cumbersome to carry out in source-limited situations.

In contrast, a passive approach for sensitivity improvement that requires minimal modification of the bioassay, little or no energy input, and no augmentation of the original instrumentations and operations is desirable. To this effect, a segregative phase separation system (SPSS) is presented based on an aqueous two-phase system (ATPS) that increases the level of localization of signals for the enhancement of sensitivity. When combined with existing commercial bioassay systems, these additive compositions enhance various bioassays, including nucleic acid tests and immunoassays.

### BRIEF SUMMARY OF THE INVENTION

Embodiments of the invention are directed to a passive method for improving bioassay sensitivity by segregative phase separation and to a bioassay signal intensifier for inclusion in a bioassay competition. The bioassay composition can be available from any kit for providing a bioassay, such as commercially available kits for performing PCR, LAMP, ELISA or other commercial bioassays. The bioassay signal intensifier is an aqueous two-phase system (ATPS) that results in a plurality of phases with partitioning the signal source into one of the aqueous phases resulting in an elevated local concentration that intensifies the signal. The ATPS can be included with all or part of a bioassay composition such that it can replace a solvent component or augment another component. The ATPS includes a first water-soluble polymer and a second water-soluble polymer that separate into two aqueous phases when their concentrations are above threshold concentrations of a binodal curve. In a non-limiting embodiment, the first water-soluble polymer can be polyethylene glycol (PEG), and the second water-soluble polymer can be dextran. In another embodiment, the ATPS forms from a water-soluble polymer and a salt, where the water-soluble polymer and the salt phase separate into two aqueous phases when above threshold concentrations of a binodal curve. These mutually immiscible aqueous phases can be one bulk phase situated over a second bulk phase or as a core-shell microparticle suspended in a non-aqueous continuous phase.

Embodiments to the passive method for performing a bioassay are to combine at least a portion of a bioassay composition for performing a bioassay of a test sample in the presence of a bioassay signal intensifier. The bioassay intensifier can be added to the bioassay composition available in a typical bioassay tool, many of which are commercially available, or the bioassay intensifier can be substituted for a component of the bioassay composition to form a segregative phase separation system (SPSS) where phase separating into at least two aqueous phases allows the partitioning of the source of the signal to intensify the bioassay signal originating from one of the at least two aqueous phases. The two aqueous phases can be two bulk phases. The signal originates from one of them, and this phase can be of lesser volume such that a concentration increase of the signal's source results in an intensification of the signal. The method can form core-shell microparticles, where one aqueous phase forms the core and the other forms the shell, suspended in a non-aqueous continuous phase. The microparticle is effectively a liquid particle and is a suspended droplet, though this will be referred to as a microparticle herein. It is advantageous to have a large partitioning coefficient into one phase, such as the core phase. The core can include the signal source, and the core can be significantly less volume than the shell. As the relative size of the core decreases, the intensity of the signal increases.

### BRIEF DESCRIPTION OF THE DRAWINGS

FIGS. 1A and 1B show a process for forming an aqueous two-phase system (ATPS) by combining two different aqueous solutions and subsequent partitioning of solutes into one of the two aqueous phases.

FIG. 2 shows a dehydration process, where the components in an aqueous microparticle concentrate and phase separate after achieving a threshold concentration where separation into a core-shell structure results, where conceptual drawings reside over photos of a microparticle in that state.

FIG. 3 shows an exemplary binodal curve for a composition of two polymers that vary in concentration to passively control their distribution as either a common aqueous solution or phase-separated aqueous solutions.

FIG. 4 shows a mode of intensifying a signal based on the segregation of the signal source in a denser aqueous phase and the reduction of the volume of that phase resulting in a smaller, more intense visible signal.

FIG. 5 shows time plots of the amplified signals by: substituting an ATPS for nucleotide-free water, A1 (top curve); adding different proportions of the bioassay signal intensifying ATPS with a LAMP composition, A2 (1:4 higher amplification) and A3 (4:1 lesser amplification); and the negative control, A4.

FIG. 6 shows a drawing of a system to form digital droplets where the droplets ultimately form core-shell microparticles suspended in an oil.

FIG. 7A shows bright and dark field images of a bioassay suspension containing purified water where fluorescence is diffuse and of low intensity.

FIG. 7B shows bright and dark field images of a bioassay suspension containing an ATPS mixture rather than water, where fluorescence is of high intensity within core portions of core-shell microparticles.

FIG. 8 shows a bar chart representing the relative fluorescence intensity of the microparticles of FIG. 7B (left) with the intensifying ATPS, and FIG. 7A (right) without the bioassay intensifier.

FIG. 9A shows a bright and dark field image of the core-shell microparticles from a PEG shell about a dextran core where the proportion of shell to core is 7.5:0.5 leading to a core-to-shell diameter ratio of 0.366 that provides a very high intensity signal.

FIG. 9B shows a bright and dark field image of the core-shell microparticles from a PEG shell about a dextran core where the proportion of shell to core is 6:2 leading to a core-to-shell diameter ratio of 0.705 that provides a much lower intensity signal than that shown in FIG. 9A.

FIG. 10 shows the preparation of phase-separated solutions to achieve a desired volume ratio of PEG-rich and Dextran-rich phases for a Quantitative Reverse Transcription Polymerase Chain Reaction (RT-qPCR) analysis.

FIG. 11 shows a plot of threshold cycles (Ct) vs plasmid concentration for use as an in-situ Calibration Curve of Plasmids in the PEG-rich phase.

FIG. 12 shows a plot of Ct vs plasmid concentration for use as an in-situ Calibration Curve of Plasmids in the Dextran-rich phase.

FIG. 13 shows a bar chart of Ct values for plasmid in PEG-rich and dextran-rich phases of ATPS with different PEG-rich to dextran-rich volume ratios.

FIG. 14 is a bar chart of the plasmid concentration in the PEG-rich and dextran-rich phases using the in situ calibration curves of FIGS. 11 and 12.

FIG. 15 is a bar chart of an enrichment factor of plasmids using ATPS at various volume ratios.

FIG. 16 shows a plot of Ct vs RNA concentration for use as an in-situ Calibration Curve of RNA in the PEG-rich phase.

FIG. 17 shows a plot of Ct vs RNA concentration for use as an in-situ Calibration Curve of RNA in the Dextran-rich phase.

FIG. 18 shows a bar chart of Ct values for RNA in PEG-rich and dextran-rich phases of ATPS with different PEG-rich:dextran-rich volume ratios.

FIG. 19 is a bar chart of the RNA concentration in PEG-rich and dextran-rich phases using the in situ calibration curves of FIGS. 16 and 17.

FIG. 20 is a bar chart of an enrichment factor of plasmids using ATPS at various volume ratios.

### DETAILED DISCLOSURE OF THE INVENTION

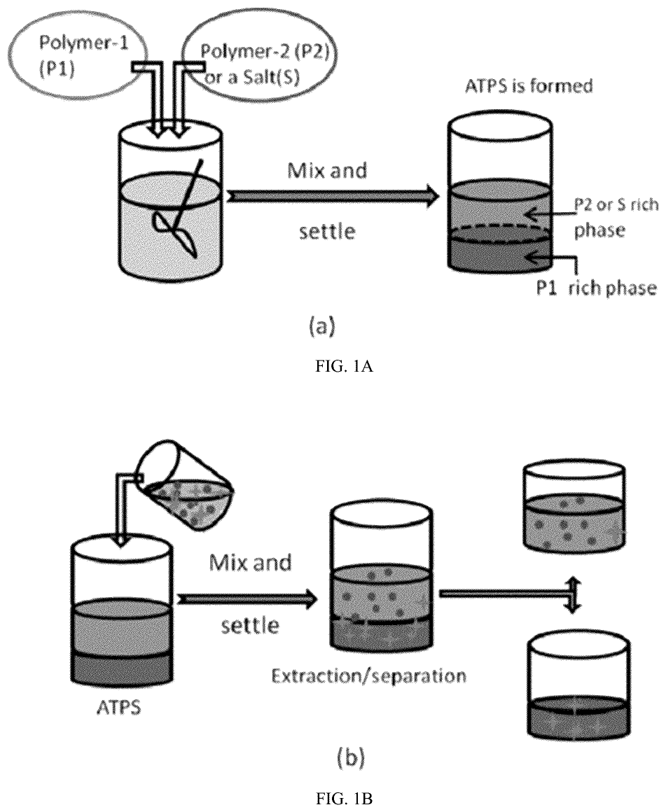

Embodiments are directed to compositions that form a segregative phase separation system (SPSS) that improves bioassay sensitivity due to a concentration increase within one phase during phase separation. This composition includes a passive bioassay signal intensifier that can be added or substituted into the composition of any bioassay system. An exemplary, by not limiting, the system is a digital droplet loop-mediated isothermal amplification (ddLAMP) bioassay. The modifying composition forms an aqueous two-phase system (ATPS) when this bioassay signal intensifier is substituted for the aqueous base of a ddLAMP mixture. This substitution requires no modification of other steps in a ddLAMP process. An ATPS is one where two agents dissolved in water cause phase separation to two aqueous solutions based on the immiscibility of the two agents. As shown in FIGS. 1A and 1B, the agents can be two immiscible polymers or a polymer immiscible in a salt solution, the second agent. The ATPS phase separation is commonly exploited for extraction or separation of products. The preferential partitioning of a biological product resulting from a bioassay protocol allows sensitivity enhancement by including the bioassay signal intensifier.

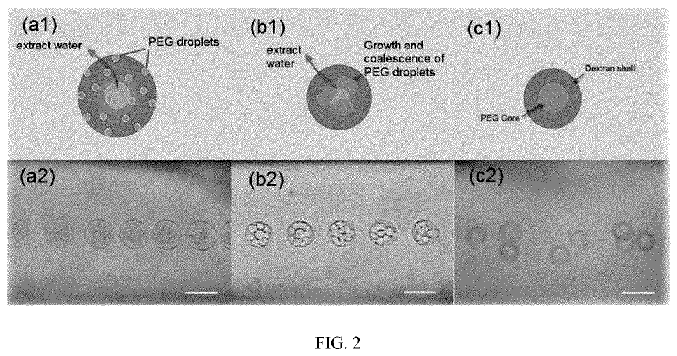

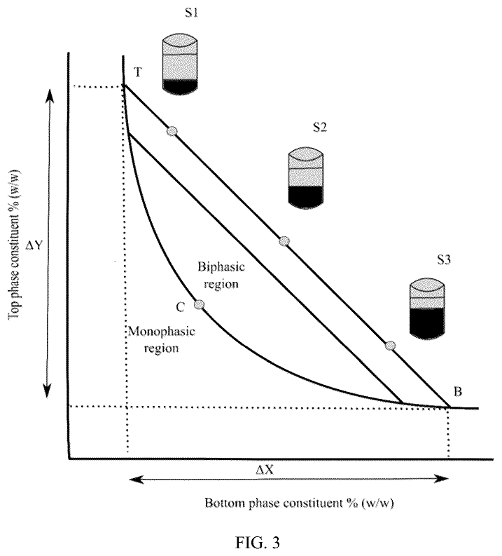

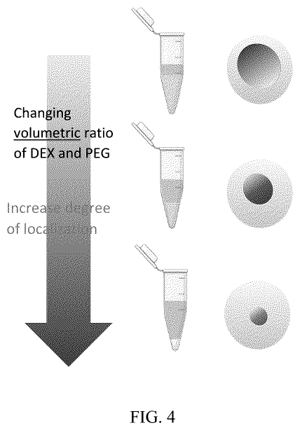

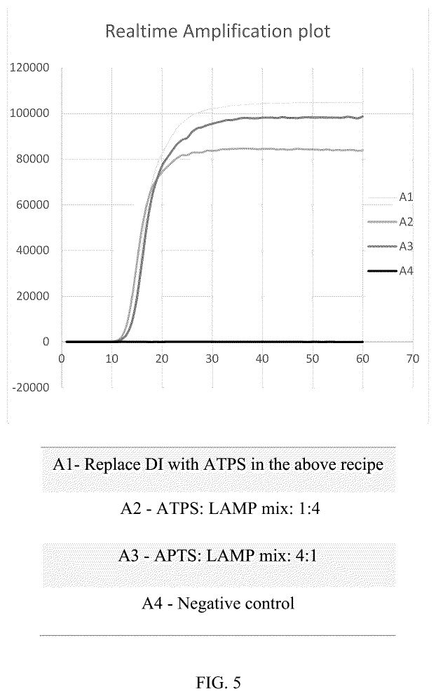

In a ddLAMP, after thermal cycling, some time is allowed wherein aqueous droplets within an oil phase undergo further phase separation to yield a core-sell two-phase structure before measuring their fluorescence. As illustrated in FIG. 2, dehydration of a combined aqueous solution or suspension, by extracting water from the droplet particle, promotes phase separation to the core-shell structure to partition and concentrate signaling reagent into one phase. The fluorescence signal concentrates in the core (or shell) phase with a higher partition affinity. In a non-limiting model system, dextran and PEG are used to generate the ATPS system where the fluorescence, due to a higher partition coefficient, resides in a Dextran core phase. Because the signal is localized inside the core phase instead of the whole droplet, the signal intensifies for the droplet. As indicated in FIG. 3, by tuning the volumetric ratio of PEG-rich and Dextran-rich phases, the bioassay signal intensifying ATPS composition can be maintained on a common tie line in the binodal curve diagram, allowing the core dextran-rich phase volume to be very small compared to the PEG-rich phase shell phase for a given partitioning coefficient. For example, in an ATPS whose volumetric ratio of dextran-rich and PEG-rich phase is below 1:9, the signal can be localized in the smaller volume, 10% of the original droplet size, such that signals that would be weak, or even undetectable, are readily detected due to the higher concentration in the smaller volume. According to an embodiment, the concentration in a core of a core-shell particle is equivalent in viewing appearance to the partitioning between two immiscible phases where, because of a conical-shaped container, the increase of concentration in the lower phase with the reduction of volume in the denser phase appears as a smaller cross-section, as shown in FIG. 4.

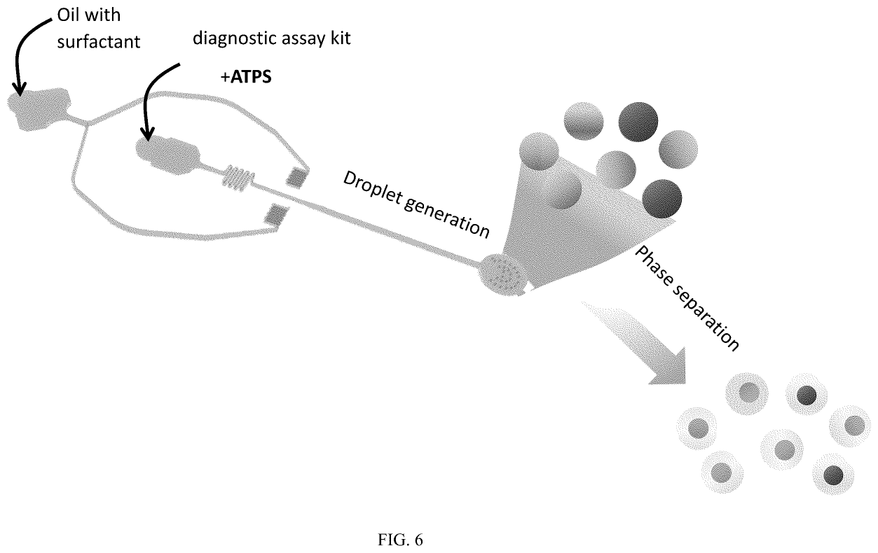

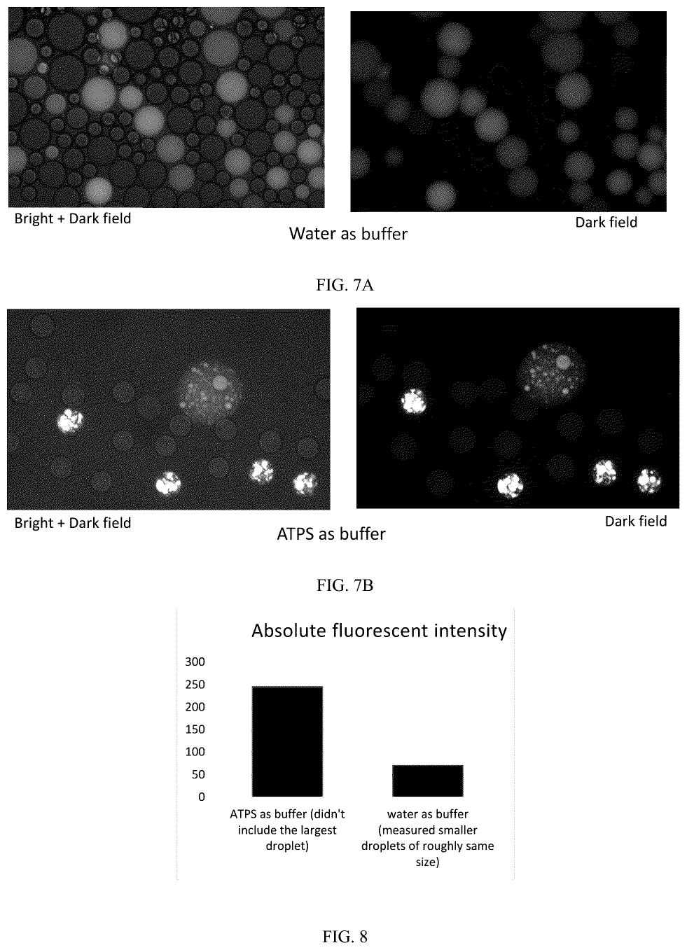

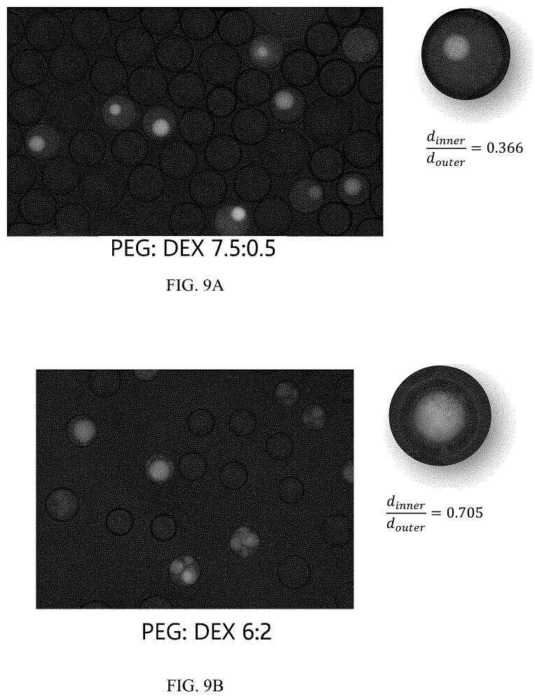

As in the ddLAMP composition modification, according to embodiments, other bioassay methods can be enhanced by segregative phase separation systems (SPSSs) employing any appropriate partition affinity depending on the composition of the specific ATPS system employed for a given bioassay. The droplet and phase separation within a droplet is illustrated in FIG. 6. By substituting a bioassay signal intensifying ATPS for the water in a bioassay composition, as illustrated in FIGS. 7A and 7B, the intensification of the fluorescent signal can be readily observed, where the absolute intensity increases more than five-fold, as shown in the bar chart of FIG. 8 for exemplary bioassays with and without the ATPS substitution for water. By varying the proportions of the ATPS components, as shown in FIGS. 9A and 9B, the size and fluorescence intensity of the core are altered, where a smaller proportion of the dextran core forming component results in a smaller core-to-shell diameter that results in the greater fluorescence intensity. Various SPSS compositions can be used to promote the intensification of various bioassays, for example, nucleic acid tests that include, but are not limited to, PCR, RT-PCR, and immunoassays, such as, but not limited to, enzyme-linked immunosorbent assay (ELISA) and their digital compartments. In the manner of the PEG-dextran system, disclosed above, any aqueous-based chemical composition promoting SPSS can be used; the composition and method are not limited to PEG and dextran but is general to any aqueous-based system that can undergo liquid-liquid phase separation (LLPS) characterized by a large partitioning coefficient contrast for a target chemical into one phase. The passive nature of this method minimizes energy input and enhances the control of the assay. This allows the application to almost any of the current bioassay kits, panels, and methods. Hence this minimal modification to existing systems and methods enhances assay without significant augmentation of systems or instruments and requires little technical training to conduct rapid process validation as typically required. Therefore, this system and method using a passive bioassay signal intensifier allow a quick and practical improvement of current bioassay techniques.

Furthermore, because different concentrations of SPSS that sit on the same tie-line yield compositions after phase separation differ only in their volumetric ratio but not the quantity of signal, a high intensification of the signal is possible. With an optimal partition affinity, a decrease in the volume of the phase containing the signal source becomes a small factor in the performance of the system.

By combining commercial assays that have proven efficacies in these SPSS, minimal additives, for example, only the bioassay signal intensifier, are introduced to the bioassay, and no extra instruments nor operations are introduced, allowing the downstream detection to remain unchanged except the increased sensitivity from that of the unmodified assay. By using various compositions of the PEG to dextran, the fluorescence signal amplification can be modified and optimized. As shown in FIG. 6, the substitution of an ATPS mixture, 1:1 PEG:Dextran at 10% by weight, for deionized (DI) water or by addition of this mixture to the LAMP composition in various exemplary real-time LAMP analyses, where two phases separating in a conical container leads to large signal amplification.

Other nucleic acid tests and antigen tests that may be combined with the current method include, but not limited to, Nucleic Acid Amplification Tests (NAATs), Reverse Transcription Polymerase Chain Reaction (RT-PCR), Nicking Endonuclease Amplification Reaction (NEAR), Transcription Mediated Amplification (TMA), Loop-Mediated Isothermal Amplification (LAMP), Helicase-Dependent Amplification (HDA), Clustered Regularly Interspaced Short Palindromic Repeats (CRISPR), and Strand displacement amplification (SDA).

In an embodiment, an ATPS-based method enhances Quantitative Reverse Transcription Polymerase Chain Reaction (RT-qPCR) assays. In this ATPS system, the plasmids of the RT-qPCR test are concentrated efficiently in a dextran-rich phase, for example, during testing for a SARS-CoV-2 Positive Control. In these tests, a high volume ratio of the PEG to Dextran phases can result in a high enrichment factor.

Following are examples that illustrate procedures for practicing the invention. These examples should not be construed as limiting. All percentages are by weight, and all solvent mixture proportions are by volume unless otherwise noted.

### Materials and Methods

The polymers compositing of the SPSS were purchased from Sigma-Aldrich and Aladdin. Bioassay kits and LAMP kits were purchased from Thermo Fisher and Guangzhou Double Helix Gene Technology Co., Ltd.

Preparation of Solution

Aqueous solutions of 10% PEG 35000 kDa and 10% Dextran 500T are prepared and well-mixed for at least half an hour to form a turbid suspension using polymer concentrations above the binodal curve in the phase diagram of the PEG-dextran system. The suspension was spun in a centrifuge at 8000 rpm for 40 minutes resulting in a dextran-rich lower phase and a PEG-rich upper phase. Cooling the mixture in a refrigerator overnight resulted in complete phase separation. Using a syringe, 30 μl of the top PEG-rich phase and 3 μl dextran-rich phase were removed separately and mixed. This solution was set aside while the bioassay was prepared. A LAMP mixture, whose original total volume was 15 μl including the template, had its 8 μl is ultra-pure water replaced with the 1:10 dextran-rich to PEG-rich solution. In this way, the PEG-dextran solution was diluted by the LAMP mixture hence the concentration of PEG and dextran decreased below the binodal curve to generate a homogenous solution lacking any phase separation, and the LAMP mixture functioned normally, as that without the substitution.

Performing ddLAMP

SPSS ddLAMP was performed according to the normal ddLAMP process with the inclusion of the bioassay signal intensifying ATPS. The SPSS-modified LAMP mixture was infused into a conventional flow-focusing microfluidic device, and the mixture was compartmentalized into pico-liter droplets. The collected droplets are placed in a microtube and put in a thermal cycler. After 63° C. incubation for 40 minutes, the droplets were collected and held for a while, during which the droplets underwent phase separation to concentrate and localize the fluorescent signal. The fluorescent signal from LAMP prefers the dextran-rich phase more than the PEG-rich phase, hence we observed that the signal is concentrated and localized in the dextran-rich core in phase-separated droplets. This partitioning and phase separation-induced signal localization could enhance the intensity of fluorescence for later detection.

Enhanced the Quantitative Reverse Transcription Polymerase Chain Reaction (RT-qPCR)

Reagents and Methods:

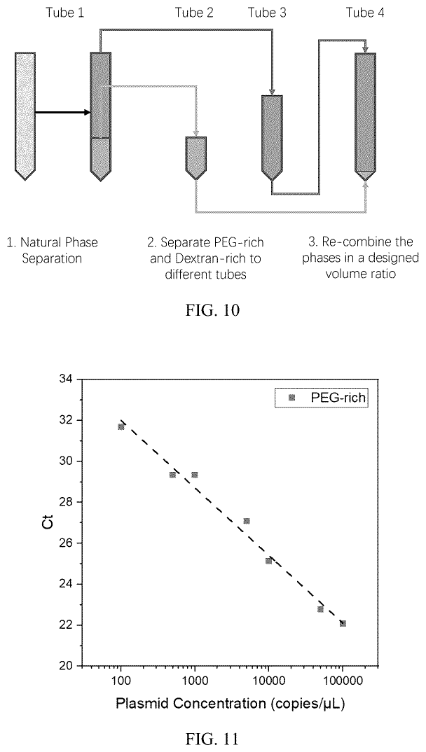

ATPS: 8% w/w PEG 8000 Da, 12% w/w Dextran 10000 Da, 80% Ultrapure Water for the stock solution. The solution preparation process is illustrated in FIG. 10.

Targets: a. SARS-CoV-2 Positive Control (N gene) (Plasmids that contain N-gene of SARS-CoV-2, purchased from New England Biolabs Inc.); and b. Viral RNA purified from the heat-inactivated lab-cultured SARS-CoV-2.

RT-qPCR: Standard RT-qPCR based on N1 and N2 targets was used to quantitatively compare the virus load in single-phase and enriched two-phase solutions. RT-qPCR was performed using Luna Probe One-Step RT-qPCR 4× Mix with UDG using the following primers and probes: (1) 2019-nCoV_N1: the forward primer 5′ GAC CCC AAA ATC AGC GAA AT 3′ and the reverse primer 5′ TCT GGT TAC TGC CAG TTG AAT CTG 3′; the 2019-nCoV_N1 Probe 5′ HEX-ACC CCG CAT TAC GTT TGG ACC-Q 3′; (2) 2019-nCoV_N2: the forward primer 5′ TTA CAA ACA TTG GCC GCA AA 3′; the reverse primer 5′ GCG CGA CAT TCC GAA 3′; the 2019-nCoV_N2 Probe 5′ 6-FAM-ACA ATT TGC CCC CAG CGC TTC AG-Q 3′. All the PCR tests were carried out using the Bio-Rad CFX Opus 96 system with a carryover prevention step of 25° C. for 30 seconds, a reverse transcription step of 55° C. for 10 minutes, and an initial denaturation step of 95° C. for 1 minute followed by 45 cycles with a denaturation step of 95° C. for 10 seconds and an extension step of 60° C. for 30 seconds.

Results:

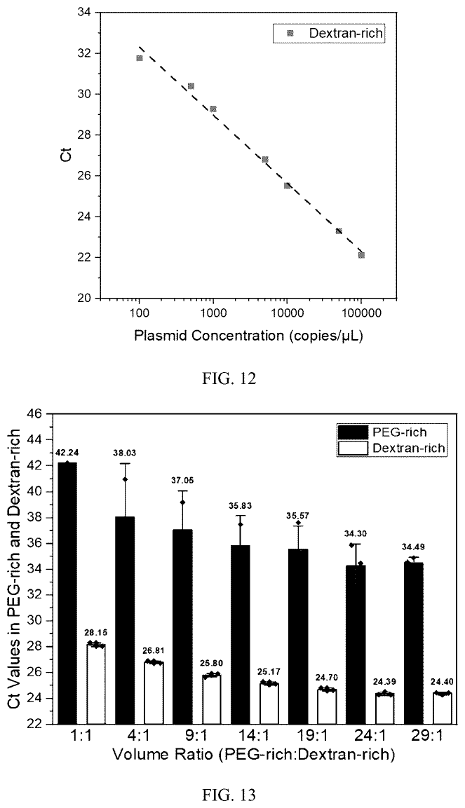

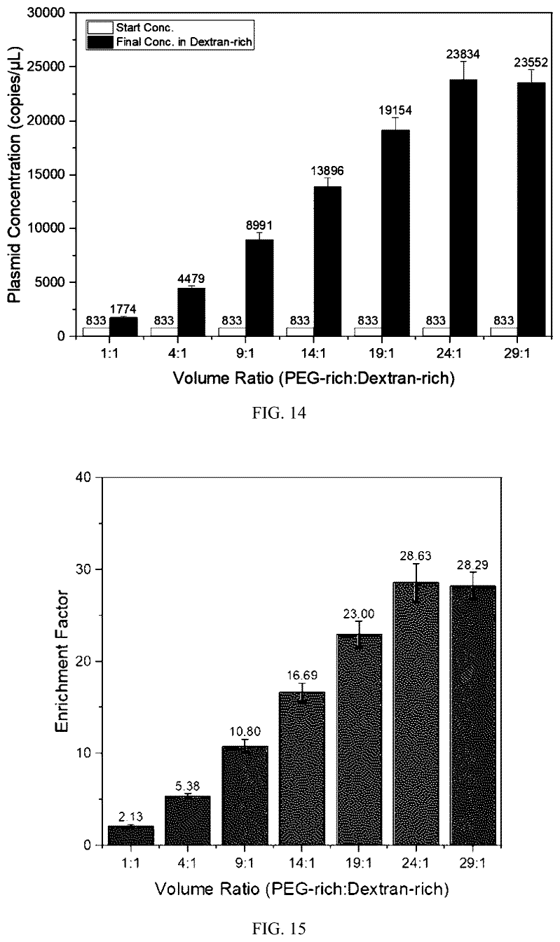

Plasmids: Two calibration curves were measured using plasmid dilutions in PEG-rich and dextran-rich phases, as shown in FIGS. 11 and 12, where these curves were used to calculate subsequent unknown plasmid concentrations. The two-phase volume ratios (PEG-rich to Dextran-rich) were adjusted to 1:1, 4:1, 9:1, 14:1, 19:1, 24:1, and 29:1. In each group, the initial plasmid concentration was 833 cp/μL where after phase separation, the ATPS enriched the plasmids partitions into the Dextran-rich phase. The threshold values (Ct), as plotted in FIG. 13, of the top PEG-rich and bottom Dextran-rich phases were evaluated using RT-qPCR. Using the calibration curves shown in FIGS. 11 and 12, the specific plasmid concentrations in each phase were determined, which is plotted in FIG. 14. An enrichment factor, the ratio of the final plasmid concentration in the dextran-rich phase to the initial concentration was constructed from the data in FIG. 14 for every phase ratio examined, as shown in FIG. 15. Clearly from these results, plasmids are concentrated efficiently in the dextran-rich phase, where a higher volume ratio of the PEG to dextran rich phases results in a high enrichment factor of 28.29 with a 29:1 volume ratio at a total ATPS volume of 1200 μL where the beginning concentration was 833 copies/μL.

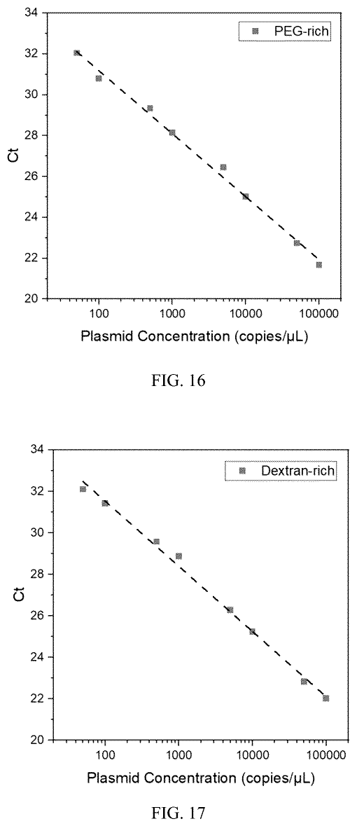

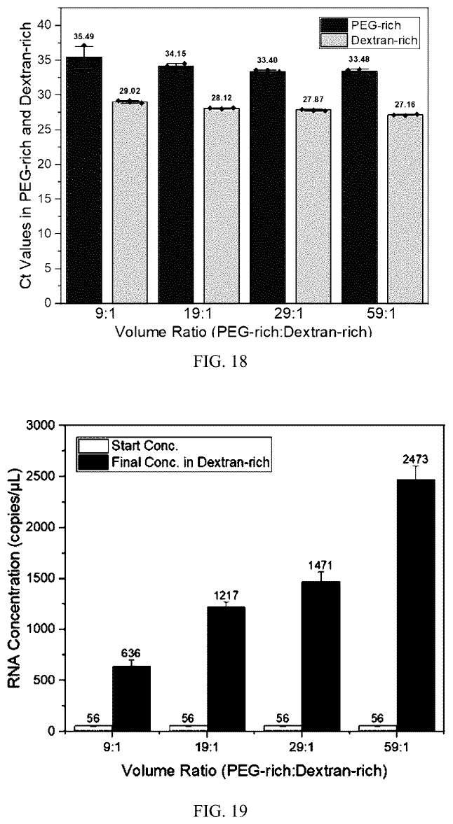

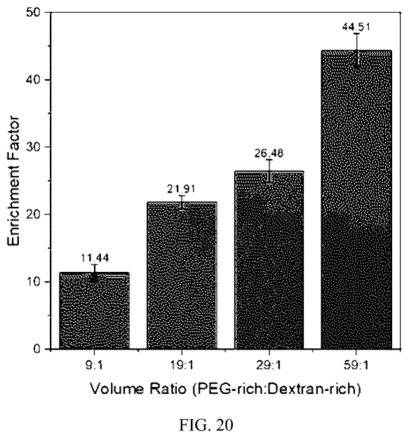

Purified RNAs: Two calibration curves were measured using RNA dilutions in PEG-rich and dextran-rich phases, as shown in FIGS. 16 and 17, where these curves were used to calculate subsequent unknown RNA concentrations. The two-phase volume ratios (PEG-rich to Dextran-rich) were adjusted to 9:1, 19:1, 29:1 and 59:1. In each group, the initial concentration was 55.55 cp/μL where after phase separation, the ATPS enriched the RNA partitions into the Dextran-rich phase. Ct values, as plotted in FIG. 18, were evaluated for the top PEG-rich and bottom Dextran-rich phases using RT-qPCR. Using the calibration curves shown in FIGS. 16 and 17, the specific RNA concentrations in each phase are plotted in FIG. 19. An enrichment factor, the ratio of the final RNA concentration in the dextran-rich phase to the initial concentration was constructed from the data in FIG. 19 for every phase ratio examined, as shown in FIG. 20. RNA is efficiently concentrated in the dextran-rich phase, where a higher volume ratio of the PEG to dextran-rich phases results in a high enrichment factor of 44.5 with a 59:1 volume ratio at a total ATPS volume of 1200 μL for the beginning concentration was 55.55 copies/μL.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Wijethunga, P. A., & Moon, H. (2015). On-chip aqueous two-phase system (ATPS) formation, consequential self-mixing, and their influence on drop-to-drop aqueous two-phase extraction kinetics. Journal of Micromechanics and Microengineering, 25(9), 094002.
Iqbal, M., Tao, Y., Xie, S., Zhu, Y., Chen, D., Wang, X., . . . & Yuan, Z. (2016). Aqueous two-phase system (ATPS): an overview and advances in its applications. Biological procedures online, 18(1), 1-18.
Song, Y., & Shum, H. C. (2012). Monodisperse w/w/w double emulsion induced by phase separation. Langmuir, 28(33), 12054-12059.
“https://www.cdc.gov/coronavirus/2019-ncov/lab/naats.html”
https://www.cdc.gov/coronavirus/2019-ncov/lab/resources/antigen-tests-guidelines.html)

We claim:

1. A bioassay signal intensifier, comprising an aqueous two-phase system (ATPS), wherein the ATPS comprises a bioassay composition, wherein a bioassay signal is intensified by partitioning of one or more signaling components of the bioassay composition selectively into one of the aqueous phases of the ATPS.

2. The bioassay signal intensifier according to claim 1, wherein the ATPS comprises a first water soluble polymer and a second water-soluble polymer, wherein solutions of the first water-soluble polymer and the second water-soluble polymer phase separate into two phases when the first and second water-soluble polymers are at concentrations above threshold concentrations.

3. The bioassay signal intensifier according to claim 2, wherein the first water-soluble polymer comprises polyethylene glycol (PEG) and the second water-soluble polymer comprises dextran.

4. The bioassay signal intensifier according to claim 1, wherein the ATPS comprises a first water-soluble polymer and a salt, wherein a first solution of the first water-soluble polymer and a second solution of the salt phase separate when exceeding threshold concentrations.

5. The bioassay signal intensifier according to claim 1, wherein the aqueous phases comprise two bulk layers.

6. The bioassay signal intensifier according to claim 1, wherein the aqueous phases comprise cores and shells of core-shell microparticles suspended in a non-aqueous liquid.

7. The bioassay signal intensifier according to claim 1, wherein the bioassay composition comprises a kit for inclusion with PCR, RT-PCR, NAAT, LAMP, ELISA, NEAR, TMA, LAMP, HAD, CRISPR, or SDA.

8. The bioassay signal intensifier according to claim 1, wherein the kit is for inclusion with RT-qPCR.

9. A method of performing a bioassay, comprising:
providing a portion of a bioassay composition comprising a plurality of reagents for performing a bioassay of a test sample;
adding a bioassay signal intensifier according to claim 1 or substituting said bioassay signal intensifier for at least one reagent or solvent to the portion of the bioassay composition to form a segregative phase separation system (SPSS);
phase separating the segregative phase separation system into at least two phases wherein an intensified bioassay signal originates from one of the at least two phases; and
measuring the intensified signal for analyzing the test sample.

10. The method according to claim 9, wherein the bioassay composition comprises components of a kit for PCR, LAMP, or ELISA.

11. The method according to claim 9, wherein the bioassay composition comprises components of a kit for RT-qPCR.

12. The method according to claim 9, wherein the bioassay signal intensifier comprises a first water-soluble polymer and a second water-soluble polymer, wherein solutions of the first water-soluble polymer and the second water-soluble polymer phase separate into two aqueous phases when above threshold concentrations of a binodal curve.

13. The method according to claim 12, wherein the first water-soluble polymer comprises polyethylene glycol (PEG) and the second water-soluble polymer comprises dextran.

14. The method according to claim 9, wherein the bioassay signal intensifier comprises a first water-soluble polymer and a salt, wherein solutions of the first water-soluble polymer and the salt phase separate into two aqueous phases when above threshold concentrations of a binodal curve.

15. The method according to claim 9, wherein phase separating comprises forming two bulk aqueous phases wherein the intensified signal resides in one of the bulk aqueous phases.

16. The method according to claim 15, wherein the bulk aqueous phase with the intensified signal is smaller in volume than the other bulk aqueous phase.

17. The method according to claim 9, wherein phase separating comprises forming aqueous phases comprising cores and shells of core-shell microparticles suspended in a non-aqueous liquid wherein the intensified signal resides in one of the aqueous phases.

18. The method according to claim 17, wherein the aqueous phases with the intensified signal is the core.

19. The method according to claim 18, wherein the core is of lesser volume than the shell.
